(19) United States
(12) Patent Application Publication
(10) Pub. No.: US 2014/0044816 A1
(43) Pub. Date: Feb. 13, 2014

(54) SPRAY METHOD AND COMPOSITION FOR REDUCING SUBSTANCE CRAVING

(71) Applicant: Michael Cherkassky, Fort Worth, TX (US)

(72) Inventor: Michael Cherkassky, Fort Worth, TX (US)

(21) Appl. No.: 13/571,399

(22) Filed: Aug. 10, 2012

(51) Int. Cl. A61K 36/81; A61P 3/00; A61P 25/34

(52) U.S. Cl. 424/760

(57) ABSTRACT

A spray composition and method are provided that are effective for reduction or elimination of the psychological hunger for foods for which a. patient wishes to reduce consumption. Such ingredients preferably include but are not limited to cayenne pepper, alcohol, salt, citric acid and a sweetener such as saccharin The composition of the invention is preferably sprayed into the mouth, resulting in a lessening or elimination of psychological hunger,

### BACKGROUND OF THE INVENTION

1. Field of the Invention

This invention relates to preparations that reduce craving for tbod substances and craving for other substances that are orally consumed or otherwise orally .provided such as the smoking of tobacco. In particular the invention relates to spray preparations and a method for reducin g psychological oral craving.

2. Description of the Related Art

There are many physical reasons why people crave food, the most basic of which to is actual hunger and stomach pangs because of lack of eating often enough and insufficient food consumption. For persons who have eaten sufficiently however, there are also often feelings of the need to eat that can be termed “psychological hunger”. Psychological hunger generally occurs when people are bored, and their thoughts turn to food and they eat because it is something to do and is interesting. Psychological hunger also can occur when people are feeling stress, and they feel that their stress can be relieved by eating.

Excessive eating when people are bored or under stress can result in increases in weight, which can become a major problem, especially when they are already overweight or obese. Further, their excessive weight can cause them stress, causing them to eat more and exacerbating the problem. Even when people are on weight loss diets and are able to regulate their eating at mealtimes, psychological stress can cause them to eat more than their diet allows, particularly between meals if they are bored or under stress.

Today obesity is one of the most serious health problems in the United States, with approximately 30% of adults suffering from obesity, and at least 50% of adults in the United States being overweight. The problem of obesity in the United States and most western countries has been steadily increasing in the last several decades. Such obesity has caused or contributed to a marked increase, in the occurrence of heart diseases, hypertension, diabetes, arthritis and increased morbidity and mortality. There is also recent research which links obesity with different types of cancer, particularly breast cancer. Obesity is a serious public, health hazard, second in importance only to tobacco. Being overweight reduces lifespan as well as quality of life.

There are many methods suggested for management of obesity and overweight. These include diets that exclude fats and high caloric, elements, appetite suppressants, psychotherapeutic techniques and operative techniques. One of the most common methods is the use of stimulants. Amphetamine-like agents act on the brain to reduce the sensation of hunger. Experience indicates that most of the appetite suppressants work for a short period of time, but a few weeks or a few months later they lose most of their potency and patients start regaining weight. There is also a serious problem with the maintenance of a desirable weight after it is achieved, for the simple reason that appetite suppressants cannot be continued indefinitely at full strength.

The reason that most people become overweight is that they consume more nutrition calories than they require, often primarily because of cravings for certain foods, such as chocolate or potato chips. In other words, humans do not only eat, to survive, but also eat for the taste, flavor and gratification. The degree of the satiety has changed and is predicated not only the necessary nutritional requirements but also on unphysiological “unnatural” pleasure drive.

The method of Cherkassky (co-pending U.S. patent application Ser. No. 12/806,668) provides a product and a treatment method using certain food ingredients in a manner that reduces or eliminates the craving for eating of foods for which the patient wishes to reduce consumption, Such ingredients include but are not limited to margarine, mustard, pepper and salt, preferably applied to the surface of the tongue as a paste, and then the craved substance is administered orally with the paste or with the non-binder components of the paste. Because psychological hunger is otlen not related to a particular substance craving, there is a need for a composition and method that generally reduces psychological hunger.

It is therefore all object of the invention herein to provide a spray composition and method that reduces psychological hunger without being targeted toward any particular food craving.

It is an object of the invention herein to provide a non-toxic method and composition for reducing or eliminating psychological hunger without side effects It is an object of the invention herein to provide a spray composition and method that can be easily used instead of or between applications of paste to the tongue to reduce cravings for intake of food that are caused by stress and boredom.

It is a further object of the invention herein to provide a spray composition and a treatment method that reduce the craving for tobacco.

Other objects and advantages will be more full apparent from the following disclosure and appended claims.

### SUMMARY OF THE INVENTION

The invention herein is a spray composition and method effective for reduction or elimination of the psychological hunger for bonds for which a patient wishes to reduce consumption, and alternatively, for tobacco. Such ingredients preferably include but are not limited to a pepper, such as cayenne (red) pepper, alcohol, salt, citric acid and a sweetener such as saccharin The cornposition of the invention is preferably sprayed into the mouth, resulting in a. lessening or elimination of psychological hunger.

Other objects and features of the inventions will be more fully apparent from the is following disclosure and appended claims.

### DETAILED DESCRIPTION OF THE INVENTION AND PREFERRED EMBODIMENTS THEREOF

The present invention in its preferred embodiment includes the administration of the composition of the invention in the form of a spray to humans. The preferred composition of the invention includes a sharp spice, preferably any kind of dried, ground pepper, and most preferably cayenne (red) pepper as is available for cooking, alcohol, salt, citric acid and saccharin (non-calorie sweetener).

The composition of the invention is administered by spraying into the mouth as discussed herein.

The proportion of ingredients can vary, and additional ingredients may be included.

The composition of the invention is preferably prepared by mixing the ingredients to form a sprayable liquid.

In addition, the composition of the invention herein for reducing craving can be combined with currently commercially available or future preparation without detrimental effect because of the lack of side effects of the invention herein.

Administration of the invention therefore is in the form of spraying a liquid into the mouth of a person that suffers from psychological hunger, such as may be due to boredom or stress.

As an added benefit of this type of administration according to the invention, patients who smoke may further be caused to cease or reduce smoking by encouraging the patient to spray the composition of the invention into the mouth. [is that correct?]

The features of the present invention will be more clearly understood by reference to the following examples, which are not to be construed as limiting the invention.

### EXAMPLE 1

### Preparation of Composition of the Invention

The method of preparation of the composition of the invention herein is preferably as follows:
1. Mix a pepper (preferably a dried, ground, pepper), such as the standard spice of cayenne (red) pepper with alcohol (1:10) and allow to sit covered at room temperature for approximately a week. The alcohol may be any consumable, non-toxic alcohol, such as ethanol or cooking brandy and the like
2. Remove sediment from the pepper-alcohol mixture, preferably by filtering four times through standard filter paper as known in the art.
3. To 40 cc of the supernatant liquid of step 2, add 30 cc salt (NaCl table salt).
4. Mix in 250 cc alcohol (such as cooking brandy), 12 cc citric acid and 1.25 cc saccharin (or other non-caloric sweetener).
5. Add 1000 cc isotonic salt solution (physiological saline); alternatively water may be used.
6. Add 500 cc vanilla extract (standard, off-the-shelf, as known in the art).

While the concentrations provide above are ⁻preferred, variations in the concentrations of the components can be made without causing the composition to he ineffective.

The composition of the invention is preferably stored at room temperature.

### EXAMPLE 2

### Use of the Composition of the Invention

The composition of the invention according to Example 1 is placed in a spray bottle for individual use. Persons desiring to reduce psychological hunger that may be caused by boredom or stress or other causes spray a few puffs (e.g. 2-4 puffs) of the composition of the invention into their mouths at any time of the day. There is immediate relief from the psychological hunger, which is decreased or eliminated completely. If the hunger is truly psychological hunger (and not physical hunger), such an administration of the composition of the invention gives 6-8 hours of relief.

If the person has actual physical hunger and is trying to reduce food consumption, the person may also have administration of the paste composition or Cherkassky or consumption of diet pills as known in the art.

The composition may be administered more frequently if desired.

While the invention has been described with reference to specific embodiments, it will he appreciated that numerous variations, modifications, and embodiments are possible, and accordingly, all such variations, modifications, and embodiments are to be regarded as being within the spirit and scope of the invention.

1. A method of reducing psychological hunger of a person, comprising spraying into the mouth of the person a liquid composition comprising: a cayenne pepper, a consumable alcohol, sodium chloride, citric acid, a sweetener, and vanilla extract, wherein prior to forming the liquid composition, the cayenne pepper is mixed with alcohol, the cayenne pepper alcohol mixture is allowed to sit about a week and is then filtered to remove sediment and form a sediment-free supernatant liquid, which is added to the liquid composition.

2. (canceled)

3. The method of claim 1, wherein the consumable alcohol comprises brandy.

4. The method of claim 1, wherein the sweetener is saccharin.

5. (canceled)

6. A method of reducing psychological hunger of a person, comprising:
a) preparing a liquid composition comprising: cayenne pepper, a consumable alcohol, sodium chloride, citric acid, a sweetener, and vanilla extract, wherein prior to forming the liquid composition, the cayenne pepper is mixed with alcohol, the cayenne pepper alcohol mixture is allowed to sit about a week and is then filtered to remove sediment and form a sediment-free supernatant liquid, which is added to the liquid composition; and
b) spraying the liquid composition into the mouth of the person.

7. The method of claim 6, wherein the sweetener is saccharin.

8. The method of claim 6, wherein the liquid composition is prepared by steps comprising:
a) mixing cayenne pepper with alcohol to form a mixture and allowing the mixture to sit covered;
b) removing sediment from the mixture resulting in a supernatant liquid;
c) mixing salt, alcohol, sweetener, citric acid and vanilla with the supernatant liquid; and
d) adding an aqueous liquid selected from the group consisting of isotonic salt solution and water.

9. The method of claim 6, wherein the consumable alcohol comprises brandy.

10. The method of claim 8, wherein the aqueous liquid is isotonic salt solution.

11. (canceled)

12. The method of claim 8, wherein the sweetener is saccharin.

13. A method of preparing a liquid composition for reducing psychological hunger, comprising:
a) mixing cayenne pepper with alcohol to form a mixture and allowing the mixture to sit covered;
b) removing sediment from the mixture resulting in a supernatant liquid;
c) mixing salt, alcohol, citric acid, sweetener, and vanilla with the supernatant liquid; and
d) adding to the supernatant liquid an aqueous liquid selected from the group consisting of isotonic salt solution and water to form the liquid composition, wherein spraying the liquid composition into the mouth of a person reduces psychological hunger.

14. The method of claim 13, wherein the alcohol comprises cooking brandy.

15. The method of claim 13, wherein the cayenne pepper is soaked in alcohol before being added to the alcohol, salt, citric acid, sweetener, and vanilla extract.

16. The method of claim 13, wherein the aqueous liquid is an isotonic salt solution.

17. The method of claim 13, wherein the sweetener is saccharin.

18. The method of claim 15, wherein the sweetener is saccharin.

19. A liquid composition for reducing psychological hunger of a person comprising: cayenne pepper; alcohol; salt; citric acid; saccharin; vanilla extract; and an aqueous liquid.
